(19) United States
(12) Patent Application Publication
(10) Pub. No.: US 2003/0171665 A1
(43) Pub. Date: Sep. 11, 2003

(54) Image space correction for multi-slice helical reconstruction

(76) Inventor: Jiang Hsieh, Brookfield, WI (US)

Correspondence Address: John S. Beulick, Armstrong Teasdale LLP, Suite 2600, One Metropolitan Square, St. Louis, MO, 63102-2740, US

(21) Appl. No.: 10/091,680

(22) Filed: Mar. 5, 2002

(51) Int. Cl. A61B 5/05; A61B 8/00

(52) U.S. Cl. 600/407; 600/437

(57) ABSTRACT

A method for facilitating reconstruction of an image includes estimating a gradient for at least one high-density object, generating a gradient image using the estimated gradient, and generating an error-candidate projection using the gradient image.

### BACKGROUND OF THE INVENTION

This invention relates to computed tomographic (CT) imaging, and more particularly to methods and apparatus for reducing imaging artifacts in an image generated using a multi-slice CT imaging system.

Phantom and clinical studies have shown that severe image artifacts can result when certain parts of an anatomy are scanned. For example, “pin-wheel” or “bear-claw” image artifacts are often produced around patient ribs or spines. These artifacts are caused mainly by a rapid change of the anatomy in the z direction and an inability of a linear interpolation to produce accurate sample-estimation.

### BRIEF DESCRIPTION OF THE INVENTION

In one aspect, a method for facilitating reconstruction of an image is provided. The method includes estimating a gradient for at least one high-density object, generating a gradient image using the estimated gradient, and generating an error-candidate projection using the gradient image.

In another aspect, a computer is programmed to estimate a gradient for at least one high-density object, generate a gradient image using the estimated gradient, and generate an error-candidate projection using the gradient image.

In yet another embodiment, a computed tomographic (CT) imaging system for reconstructing an image of an object is provided. The imaging system includes a detector array, at least one radiation source, and a computer coupled to the detector array and the radiation source. The computer is configured to estimate a gradient for at least one high-density object, generate a gradient image using the estimated gradient, and generate an error-candidate projection using the gradient image.

### BRIEF DESCRIPTION OF THE DRAWINGS

FIG. 1 is a pictorial view of a CT imaging system.

FIG. 2 is a block schematic diagram of the system illustrated in FIG. 1.

FIG. 3 illustrates generated images of a patient scan.

FIG. 4 illustrates a plurality of images of a patient scan corrected with masking.

### DETAILED DESCRIPTION OF THE INVENTION

In some CT imaging system configurations, an x-ray source projects a fan-shaped beam which is collimated to lie within an X-Y plane of a Cartesian coordinate system and generally referred to as an “imaging plane”. The x-ray beam passes through an object being imaged, such as a patient. The beam, after being attenuated by the object, impinges upon an array of radiation detectors. The intensity of the attenuated beam radiation received at the detector array is dependent upon the attenuation of an x-ray beam by the object. Each detector element of the array produces a separate electrical signal that is a measurement of the beam attenuation at the detector location. The attenuation measurements from all the detectors are acquired separately to produce a transmission profile.

In third generation CT systems, the x-ray source and the detector array are rotated with a gantry within the imaging plane and around the object to be imaged so that the angle at which the x-ray beam intersects the object constantly changes. A group of x-ray attenuation measurements, i.e., projection data, from the detector array at one gantry angle is referred to as a “view”. A “scan” of the object comprises a set of views made at different gantry angles, or view angles, during one revolution of the x-ray source and detector.

In an axial scan, the projection data is processed to construct an image that corresponds to a two dimensional slice taken through the object. One method for reconstructing an image from a set of projection data is referred to in the art as the filtered back projection technique. This process converts the attenuation measurements from a scan into integers called “CT numbers” or “Hounsfield units”, which are used to control the brightness of a corresponding pixel on a cathode ray tube display.

To reduce the total scan time, a “helical” scan may be performed. To perform a “helical” scan, the patient is moved while the data for the prescribed number of slices is acquired. Such a system generates a single helix from a one fan beam helical scan. The helix mapped out by the fan beam yields projection data from which images in each prescribed slice may be reconstructed.

Reconstruction algorithms for helical scanning typically use helical weighing algorithms that weight the collected data as a function of view angle and detector channel index. Specifically, prior to a filtered back projection process, the data is weighted according to a helical weighing factor, which is a function of both the gantry angle and detector angle. The helical weighting algorithms also scale the data according to a scaling factor, which is a function of the distance between the x-ray source and the reconstruction plane. The weighted and scaled data is then processed to generate CT numbers and to construct an image that corresponds to a two dimensional slice taken through the object. Alternatively, projections can be first interpolated to produce a set of new projections prior to the filtered backprojection. Other helical reconstruction algorithms can also be used.

Phantom and clinical studies have shown that severe image artifacts can result when certain part of the anatomy is scanned. For example, “pin-wheel” or “bear-claw” image artifacts are often produced around patient ribs or spines. These artifacts are caused mainly by the rapid change of the anatomy in the z direction and the inability of the linear interpolation to produce accurate sample-estimation.

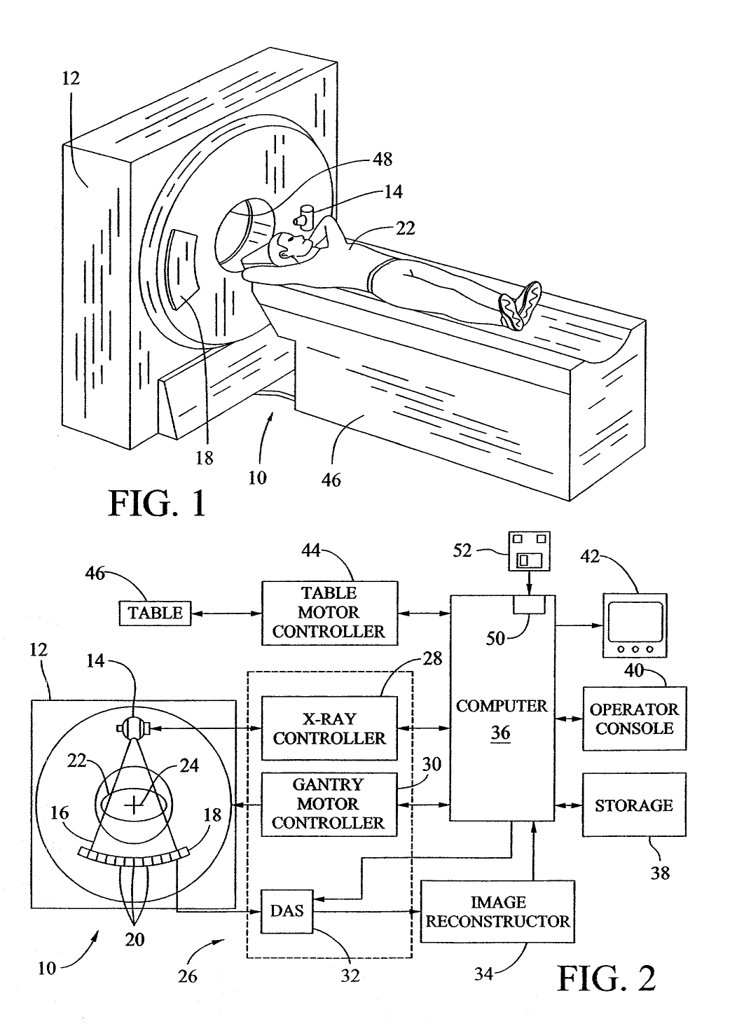

Referring to FIGS. 1 and 2, a multi-slice scanning imaging system, for example, computed tomography (CT) imaging system 10, is shown as including a gantry 12 representative of a “third generation” CT imaging system. Gantry 12 has an x-ray source 14 that projects a beam of x-rays 16 toward a detector array 18 on the opposite side of gantry 12. Detector array 18 is formed by a plurality of detector rows (not shown) including a plurality of detector elements 20 which together sense the projected x-rays that pass through an object, such as a medical patient 22. Each detector element 20 produces an electrical signal that represents the intensity of an impinging x-ray beam and hence the attenuation of the beam as it passes through object or patient 22. During a scan to acquire x-ray projection data, gantry 12 and the components mounted thereon rotate about a center of rotation 24. FIG. 2 shows only a single row of detector elements 20 (i.e., a detector row). However, multislice detector array 18 includes a plurality of parallel detector rows of detector elements 20 so that projection data corresponding to a plurality of quasi-parallel or parallel slices can be acquired simultaneously during a scan.

Rotation of gantry 12 and the operation of x-ray source 14 are governed by a control mechanism 26 of CT system 10. Control mechanism 26 includes an x-ray controller 28 that provides power and timing signals to x-ray source 14 and a gantry motor controller 30 that controls the rotational speed and position of gantry 12. A data acquisition system (DAS) 32 in control mechanism 26 samples analog data from detector elements 20 and converts the data to digital signals for subsequent processing. An image reconstructor 34 receives sampled and digitized x-ray data from DAS 32 and performs high-speed image reconstruction. The reconstructed image is applied as an input to a computer 36 which stores the image in a mass storage device 38.

Computer 36 also receives commands and scanning parameters from an operator via console 40 that has a keyboard. An associated cathode ray tube display 42 allows the operator to observe the reconstructed image and other data from computer 36. The operator supplied commands and parameters are used by computer 36 to provide control signals and information to DAS 32, x-ray controller 28 and gantry motor controller 30. In addition, computer 36 operates a table motor controller 44 which controls a motorized table 46 to position patient 22 in gantry 12. Particularly, table 46 moves portions of patient 22 through gantry opening 48.

In one embodiment, computer 36 includes a device 50, for example, a floppy disk drive or CD-ROM drive, for reading instructions and/or data from a computer-readable medium 52, such as a floppy disk or CD-ROM. In another embodiment, computer 36 executes instructions stored in firmware (not shown). Computer 36 is programmed to perform functions described herein, but other programmable circuits can be likewise programmed. For example, in one embodiment, DAS 32 performs functions described herein. Accordingly, as used herein, the term computer is not limited to just those integrated circuits referred to in the art as computers, but broadly refers to computers, processors, microcontrollers, microcomputers, programmable logic controllers, application specific integrated circuits, and other programmable circuits. Additionally, although described in a medical setting, it is contemplated that the benefits of the invention accrue to all CT systems including industrial CT systems such as, for example, but not limited to, a baggage scanning CT system typically used in a transportation center such as, for example, but not limited to, an airport or a rail station.

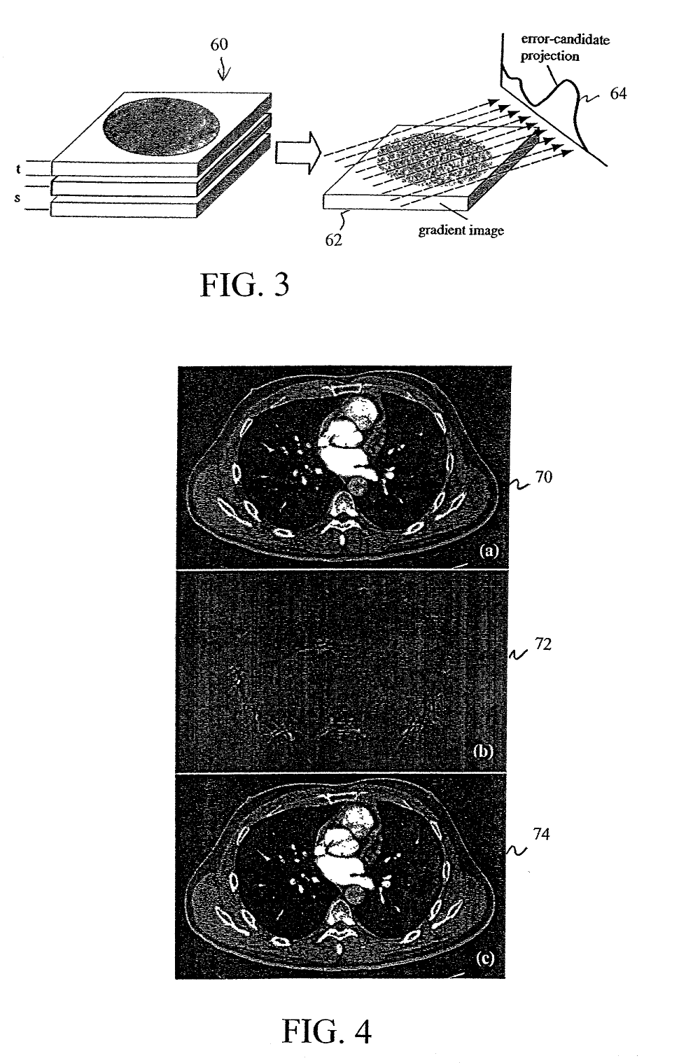

FIG. 3 illustrates three images 60 produced with a conventional reconstruction algorithm. Three images 60 are adjacent to each other with a spacing s. The spacing s is advantageously selected to facilitate a good estimate of the variation of high-density anatomies in any given area. For example, s is selected to be equal to a nominal slice thickness of the reconstructed image, t, in one embodiment. In other embodiments, s is not equal to the nominal slice thickness. The reconstructed images are labeled f₋, f, and f₊, respectively. Three high-density images (d₋, d, and d₊) are generated based on the reconstructed pixel intensities. For example, using a thresholding technique, all the pixels with intensities less than the threshold, h, are set to zero:

$$\begin{matrix}{d}_{-} ( i , j ) = \{ \begin{matrix}{f}_{-} ( i , j ) - h , & {f}_{-} ( i , j ) \geq h \\ 0 & otherwise\end{matrix} \\ d ( i , j ) = \{ \begin{matrix}f ( i , j ) - h , & f ( i , j ) \geq h \\ 0 & otherwise\end{matrix} \\ {d}_{+} ( i , j ) = \{ \begin{matrix}{f}_{+} ( i , j ) - h , & {f}_{+} ( i , j ) \geq h \\ 0 & otherwise\end{matrix}\end{matrix}$$

A gradient image 62 (g), is computed which represents the variation of the high-density object in z at the reconstructed image location:

g(i,j)=d₋(i,j)+d+(i,j)−2d(i,j)

In other embodiments, other equations are used to estimate the gradient of the high-density objects. After gradient image 62 is produced, an error-candidate projection 64 is generated based on the weighted gradient image, as shown in FIG. 3. An error-candidate image is generated by weighting a plurality of error-candidate projections with a scaling factor. The scaling changes with the relative position between the actual detector and the plane-of-reconstruction. The scaling is also dependent on the type of helical weight used in the original reconstruction to produce images f₋, f, and f₊. In one embodiment, the scaling is also a function of the detector angle, γ. To simplify the calculation, one can approximate the helical weight by the average weight for the entire projection and assign the scaling factor based on the averaged helical weight for that projection. Alternatively, one can use the helical weight applied to the iso-channel to approximate the helical weighting function.

For illustration, and considering a specific example in which the “center view” of the scan is defined as the projection angle at which the detector center and the plane-of-reconstruction overlap. The detector center is located mid-point between detector rows M/2 and (M/2)+1 for an M-row detector (wherein the first row is row number 1). Denote β_{c} as the center view angle, p the helical scan pitch, and N the number of views per gantry rotation, the z location of the plane-of-reconstruction relative to the detector center for projection view β will be:

$$z = \frac{( β - {β}_{c} ) p}{2 π} + \frac{M + 1}{2}$$

In this notation, the z locations for the M detector rows are: 1, 2, 3, . . . , M. If the helical weight is linear interpolation based, the projection error can be assumed to be roughly proportional to the distance of the plane-of-reconstruction to the nearest detector row. For example, if the plane-of-reconstruction overlaps with one of the detector row sample, the projection error is zero. The largest error occurs when the plane-of-reconstruction is straddled between two detector rows. The error-fraction, c_{β}, is calculated such that:

c_{β}=z−int(z)

where int(z) is the integer portion of z, and the subscript β denotes the projection view angle at which the error-fraction is calculated. FIG. 4 illustrates an example of the error-fraction as a function of projection angle for an eight-slice scanner. Of course, other higher-order models can be used to estimate the projection error based on the distance. Using the gradient image, g, and the error-fraction, c_{β}, the error-candidate projection, Pₑ, is produced by forward projecting the gradient image along β and scaling the projection by the error-fraction. In one embodiment, a fan beam forward projection is performed, since the original data is acquired in fan beam mode. This facilitates that the spacing between forward-projected samples matches the original data acquisition geometry. Additionally, for computational efficiency, parallel beam projections can be used to approximate the fan beam, and the sampling spacing can be relaxed. Mathematically, the operation can be denoted by Pₑ=c_{β}·FP(g), where FP is the forward projection operator. Once the error-candidate projection is obtained, the error image, ξ, can be obtained by the reconstruction of the error-candidate projections. Also, additional helical weighting functions can be applied to the error-candidate projection prior to the filtered backprojection. The helical weighting function can be identical to the original weighting that produced the original image, or an averaged or modified version of the weights. The final image, f_{c}, can be obtained by subtracting the original image from the error image: f_{c}(i, j)=f(i,j)−k·ξ(i,j),where k is a scaling factor.

FIG. 4 illustrates reconstructed images of a patient chest scan. More specifically, FIG. 4 includes an image 70 reconstructed with one of the original algorithms. Bear-claw artifacts near the ribs are quite obvious. FIG. 4 also includes an error image 72 (ξ). It is clear that the error pattern resembles that of the original artifacts. FIG. 4 also includes a corrected image 74 (f_{c}). Significant improvement in artifact reduction can be observed in FIG. 4.

In the above illustration, a single threshold, h, was used to produce a single gradient image and a single set of error-candidate projections. However, other different thresholds can be selected to separate different classes of objects and perform the above sequence of operations on each class. The final image is then the original image subtracting the scaled error candidate images from all classes. For example, setting a very high threshold can isolate metal objects and produce an error candidate image for the metal. Also a somewhat lower threshold can be set to isolate any bony objects and produce another error candidate image for bones. Another threshold is set for contrast. And when obtaining the final image, the scaling factor, k, used for different object types is different for each threshold.

In another embodiment, an alternative method other than threshold is used. For example, a more sophisticated segmentation technique is used to separate metal, bone, and contrast to produce different gradient images.

In yet another embodiment, more than three adjacent images are used to produce the gradient image. For example, neighboring N images are used with a filtering technique to produce a gradient image.

Although particular embodiments of the invention have been described and illustrated in detail, it is to be clearly understood that the same is intended by way of illustration and example only and is not to be taken by way of limitation. In addition, the CT system described herein is a “third generation” system in which both the x-ray source and detector rotate with the gantry. However, many other CT systems including “fourth generation” systems wherein the detector is a full-ring stationary detector and only the x-ray source rotates with the gantry may be used. While the invention has been described in terms of various specific embodiments, those skilled in the art will recognize that the invention can be practiced with modification within the spirit and scope of the claims.

What is claimed is:

1. A method for facilitating reconstruction of an image, said method comprising:
estimating a gradient for at least one high-density object;
generating a gradient image using the estimated gradient; and
generating an error-candidate projection using the gradient image.

2. A method in accordance with claim 1 wherein to generate an error-candidate projection, said method further comprises forward projecting the gradient along β wherein β represents a projection view angle.

3. A method in accordance with claim 2 further comprising scaling the error-candidate projection with an error fraction based upon the β.

4. A method in accordance with claim 3 further comprising scaling the error-candidate projection with an error fraction c_{β} such that c_{β}=z−int(z),
$$z = \frac{( β - {β}_{c} ) p}{2 π} + \frac{M + 1}{2} ,$$
wherein β_{c} represents a center view angle, p is the pitch, int(z) represents the integer portion of z, and M represents the number of rows in a detector array.

5. A method in accordance with claim 2 further comprising reconstructing an error image using the error-candidate projection.

6. A method in accordance with claim 5 further comprising generating a final image by scaling the error image and subtracting the scaled error image from an original image.

7. A method in accordance with claim 1 wherein estimating a gradient for a high-density object comprises estimating a gradient for a high-density object such that g(i,j)=d₋(i,j)+d₊(i,j)−2d(i,j), where g(i,j) represents the gradient estimate for the (i,j) pixel and d₋(i,j), d₊(i,j), and d(i,j) are determined according to:
$$\begin{matrix}{d}_{-} ( i , j ) = \{ \begin{matrix}{f}_{-} ( i , j ) - h , & {f}_{-} ( i , j ) \geq h \\ 0 & otherwise\end{matrix} \\ d ( i , j ) = \{ \begin{matrix}f ( i , j ) - h , & f ( i , j ) \geq h \\ 0 & otherwise\end{matrix} \\ {d}_{+} ( i , j ) = \{ \begin{matrix}{f}_{+} ( i , j ) - h , & {f}_{+} ( i , j ) \geq h \\ 0 & otherwise\end{matrix}\end{matrix}$$
where f, f₋, and f₊ represent three images separated by a spacing s with f being between f₋ and f₊, and h is a pre-determined threshold value.

8. A method in accordance with claim 2 further comprising helically weighting the error candidate image.

9. A method in accordance with claim 2 wherein said forward projecting the gradient along β comprises performing at least one of a fan beam forward projection and a parallel beam forward projection.

10. A method in accordance with claim 1 further comprising producing different gradient images using a segmentation technique.

11. A method in accordance with claim 10 wherein said producing different gradient images using a segmentation technique comprises:
separating at least two different classes of objects including a first class and a second class;
using a first contrast threshold value for the first class; and
using a second contrast threshold value different from the first contrast threshold value for the second class.

12. A method in accordance with claim 7 further comprising using more than three adjacent images to produce a gradient image.

13. A computer programmed to:
estimate a gradient for at least one high-density object;
generate a gradient image using the estimated gradient; and
generate an error-candidate projection using the gradient image.

14. A computer in accordance with claim 13 further programmed to forward project the gradient along β wherein β represents a projection view angle.

15. A computer in accordance with claim 14 further programmed to scale the error-candidate projection with an error fraction based upon the β.

16. A computer in accordance with claim 15 further programmed to scale the error-candidate projection with an error fraction c_{β} such that c_{β}=z−int(z), where
$$z = \frac{( β - {β}_{c} ) p}{2 π} + \frac{M + 1}{2} ,$$
wherein β_{c} represents a center view angle, p is the pitch, int(z) represents the integer portion of z, and M represents the number of rows in a detector array.

17. A computer in accordance with claim 15 further programmed to reconstruct an error image using the error-candidate projection.

18. A computer in accordance with claim 17 further programmed to generate a final image by scaling the error image and subtracting the scaled error image from an original image.

19. A computer in accordance with claim 17 further programmed to perform at least one of a fan beam forward projection and a parallel beam forward projection.

20. A computer in accordance with claim 14 further programmed to estimate a gradient for a high-density object such that g(i,j)=d₋(i,j)+d₊(i,j)−2d(i,j), where g(i,j) represents the gradient estimate for the (i,j) pixel and d₋(i,j), d₊(i,j), and d(i,j) are determined according to:
$$\begin{matrix}{d}_{-} ( i , j ) = \{ \begin{matrix}{f}_{-} ( i , j ) - h , & {f}_{-} ( i , j ) \geq h \\ 0 & otherwise\end{matrix} \\ d ( i , j ) = \{ \begin{matrix}f ( i , j ) - h , & f ( i , j ) \geq h \\ 0 & otherwise\end{matrix} \\ {d}_{+} ( i , j ) = \{ \begin{matrix}{f}_{+} ( i , j ) - h , & {f}_{+} ( i , j ) \geq h \\ 0 & otherwise\end{matrix}\end{matrix}$$
where f, f₋, and f₊ represent three images separated by a spacing s with f being between f₋ and f₊, and h is a pre-determined threshold value.

21. A computer in accordance with claim 14 further programmed to:
separate at least two different classes of objects including a first class and a second class;
use a first contrast threshold value for the first class; and
use a second contrast threshold value different from the first contrast threshold value for the second class.

22. A computed tomographic (CT) imaging system for reconstructing an image of an object, said imaging system comprising:
a detector array;
at least one radiation source; and
a computer coupled to said detector array and said radiation source, said computer configured to:
estimate a gradient for at least one high-density object;
generate a gradient image using the estimated gradient; and
generate an error-candidate projection using the gradient image.

23. A CT imaging system in accordance with claim 22 wherein said computer is further programmed to forward project the gradient along β wherein β represents a projection view angle.

24. A CT imaging system in accordance with claim 23 wherein said computer is further programmed to scale the error-candidate projection with an error fraction based upon the β.

25. A CT imaging system in accordance with claim 24 wherein said computer is further programmed to scale the error-candidate projection with an error fraction c_{β} such that c_{β}=z−int(z), where
$$z = \frac{( β - {β}_{c} ) p}{2 π} + \frac{M + 1}{2} ,$$
wherein β_{c} represents a center view angle, p is the pitch, int(z) represents the integer portion of z, and M represents the number of rows in a detector array.

26. A CT imaging system in accordance with claim 25 wherein said computer is further programmed to generate a final image by scaling the error image and subtracting the scaled error image from an original image.
